(19) United States
(12) Patent Application Publication
(10) Pub. No.: US 2008/0076973 A1
(43) Pub. Date: Mar. 27, 2008

(54) REMOTE HEALTH CARE SYSTEM WITH TREATMENT VERIFICATION

(75) Inventor: Rajiv Muradia, Ottawa (CA)

Correspondence Address: MERCHANT & GOULD PC, P.O. BOX 2903, MINNEAPOLIS, MN, 55402-0903, US

(73) Assignee: Igeacare Systems Inc., Richmond Hill (CA)

(21) Appl. No.: 11/757,129

(22) Filed: Jun. 1, 2007

Related U.S. Application Data
(60) Provisional application No. 60/809,840, filed on Jun. 1, 2006.

(51) Int. Cl. A61B 5/00

(52) U.S. Cl. 600/300

(57) ABSTRACT

Disclosed are methods for remote health care treatment verification. The methods include determining a medical plan including at least a medical procedure that is self administered. The methods further include establishing a communications link, supporting the transmission of video information from the patient to a medical professional, between the medical professional and a patent remote to the medical professional. Furthermore, the methods include instructing the patient to initiate a self administered medical procedure in accordance with the medical plan and receiving video information from the patient indicative of compliance with the instruction.

### CROSS-REFERENCE TO RELATED APPLICATIONS

This patent application claims the benefit of U.S. Provisional Patent Application No. 60/809,840 entitled “REMOTE HEALTH CARE SYSTEM WITH TREATMENT VERIFICATION,” filed on Jun. 1, 2006 and expressly incorporated herein by reference.

### FIELD OF THE INVENTION

The invention relates to remote health care systems and medical plans that are administered remotely.

### BACKGROUND

Health care costs represent a significant portion of government budgets around the world. As the population ages and new expensive medical equipment and procedures are introduced health care costs continue to increase.

While many of the costs of health care are related to new technologies, it is also the case that a significant portion of health care costs are based on standard procedures. For example, in some cases it is current practice to keep a patient in a hospital to ensure that the patient follows a prescription. Alternatively, it is known to have a nurse or other medical professional arrive at the home of a patient numerous times a day simply to verify that a prescription is being followed. In the event that the patient fails to follow the prescription it is not uncommon that the consequences are severely detrimental to the health of the patient thereby incurring additional costs to the health care system. In this way, such remote visits are justified. This is a particularly difficult problem for psychiatric patients whose behavior is often difficult to predict and who frequency go to considerable lengths to avoid taking a prescription. That said, a vast majority of such patients appreciate the benefits of following a proper health care regime. These patients are content to follow their prescription.

In U.S. Pat. No. 5,908,788 by Kell, a urine test is used to verify that a patient is following their prescription by measuring the chemistry and specific gravity of the patient's urine. Clearly, such tests only provide useful information after the prescription is either provided or not. Additionally, the equipment used by Kell is not well suited to a remote health care system simply because it is relatively expensive.

It would be beneficial to provide a system that allows health care professionals to verify that patient is following a predetermined self administered health care procedure properly absent having to send a health care professional to the home of the patient or keeping the patient in a medical facility. Further, it would be beneficial if such a system were produced at a relatively low cost.

### SUMMARY

Consistent with embodiments of the present invention, systems and methods are disclosed for transmitting and processing patient physiological data derived from a self administered medical procedure at a remote location. The system and method further comprises determining a medical plan comprising at least a medical procedure that is self administered, establishing a communications link between a medical professional and a patient remote to the medical professional, the communications link supporting the transmission of video information from the patient to the medical professional; instructing the patient to initiate a self administered medical procedure in accordance with the medical plan; receiving video information from the patient indicative of compliance with the instruction.

In one embodiment, the apparatus for administering the at least one self administered medical procedure is an active sensor. The active sensor is coupled to a patient work station, which is operatively configured to determine the reliability of the communications network that facilitates transmission of patient physiological data generated from the self administered test to a health care provider analysis system. The patient work station stores patient physiological data in temporary memory and upon a determination that the communications network is reliable gradually transmits patient physiological data in an orderly fashion.

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description and include systems and methods for transmission of patient heart beat data from a patient work station to a remote health provider analysis system.

### BRIEF DESCRIPTION OF THE DRAWINGS

The invention is now described with reference to the drawings in which:

FIG. 1 is a diagram of an embodiment of the invention;

FIG. 2A. is an illustration of the process and flow of data that occurs during patient use of the system illustrated in FIG. 1;

FIG. 2B. is further illustration of the process and flow of data that occurs during patient use of the system illustrated in FIG. 1;

FIG. 3. is an illustration of the process and flow of data that occurs during care provider use of the system illustrated in FIG. 1;

FIG. 4. illustrates a login screen;

FIG. 5. illustrates an access welcome screen;

FIG. 6. illustrates a blood sugar monitoring screen;

FIG. 7. illustrates a blood sugar monitoring screen;

FIG. 8. illustrates an instruction screen;

FIG. 9. illustrates a video box;

FIG. 10. illustrates a report screen; and

FIG. 11. illustrates a graph of measurements.

### DETAILED DESCRIPTION OF THE INVENTION

The following detailed description refers to the accompanying drawings. Wherever possible, the same reference numbers are used in the drawings and the following description to refer to the same or similar parts. While several exemplary embodiments and features of the invention are described herein, modifications, adaptations and other implementations are possible, without departing from the spirit and scope of the invention. For example, substitutions, additions or modifications may be made to the components illustrated in the drawings, and the exemplary methods described herein may be modified by substituting, reordering or adding steps to the disclosed methods. Accordingly, the following detailed description does not limit the invention. Instead, the proper scope of the invention is defined by the appended claims.

The present invention relates to systems and methods in the remote health care environment. Systems and methods consistent with embodiments of the present invention support verifying compliance of a patient with a medical plan remotely. The systems and methods comprise the determining of a medical plan that includes at least a medical procedure that is self administered, the establishing of a communications link between a medical professional and a patient remote to the medical professional; a communications link supporting the transmission of video information from the patient to the medical professional; instructing the patient to initiate a self administered medical procedure in accordance with the medical plan; receiving video information from the patient indicative of compliance with the instruction. The establishment of a medical procedure that is self administered may be a patient acquiring heart beat data through use of sensor proximate the patient. In one embodiment, the sensor is an electronic stethoscope of the type that is currently available in the marketplace.

In administering the medical procedure through use of an electronic stethoscope, the patient is required to position the stethoscope audio transceivers on their body in order to facilitate capture of the heartbeat sounds for storage on the patient station and transmission to central server for analysis. During use of the electronic stethoscope, the patient may be guided by the health care provider remotely via video transmissions to the patient station which describes where the stethoscope audio transceivers are to be placed on the body. The patient may also be provided instructions on placement of the stethoscope audio transceivers by instruction positioned proximate the patient station. The patient station processes the data representative of a patient's heartbeat to determine if it is within predetermined acceptable ranges. Data representative of a patient's heartbeat that is not within acceptable ranges is flagged with an indicator so that a health care provider can easily pinpoint data which has been flagged. Next, the data representative of a patient's heartbeat along with any indicators is stored in a memory buffer. Upon a determination that the communications medium between the patient health system and the health care provider analysis system is sufficiently stable, the data within the memory buffer is transmitted in gradual and orderly fashion. Gradual and orderly data transmission helps to facilitate recovery and redistribution of incomplete data transmissions resulting from communication network service interruptions.

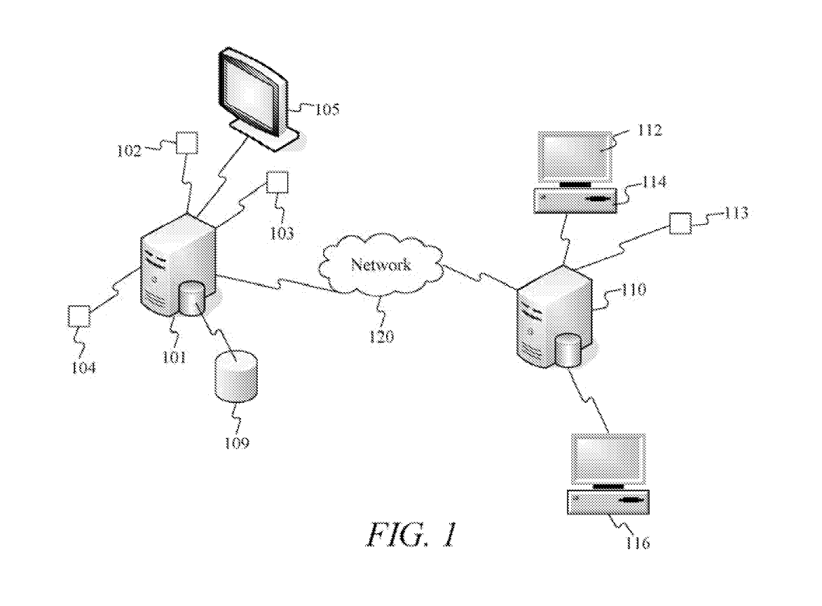

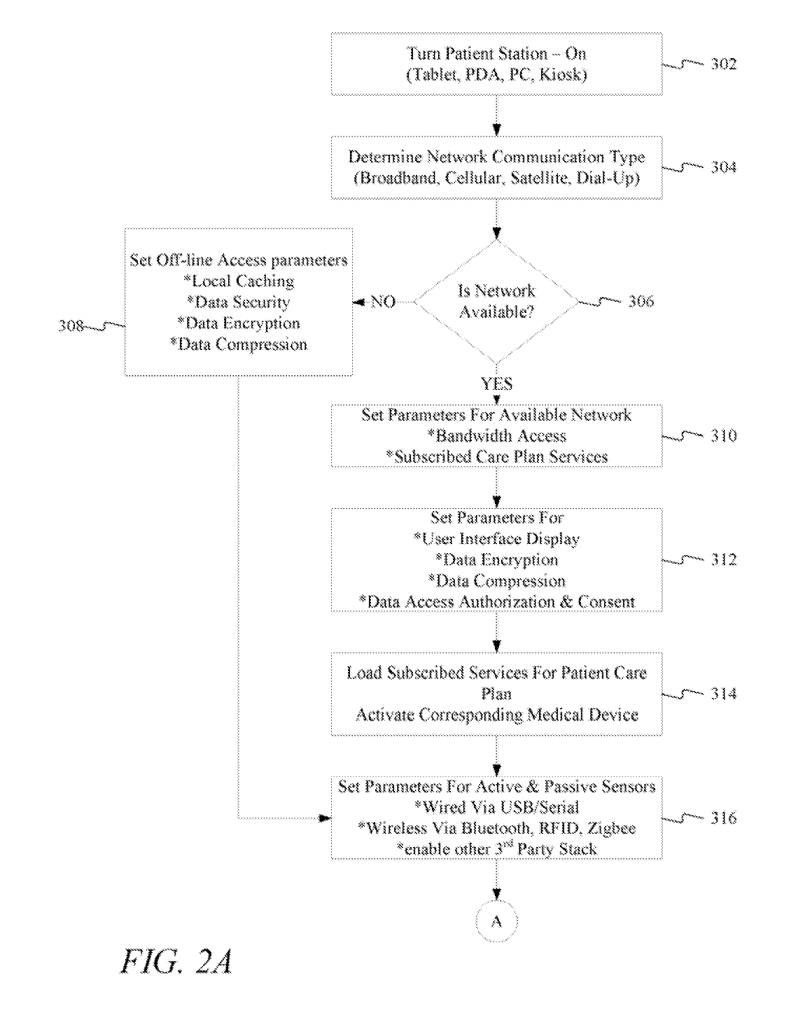

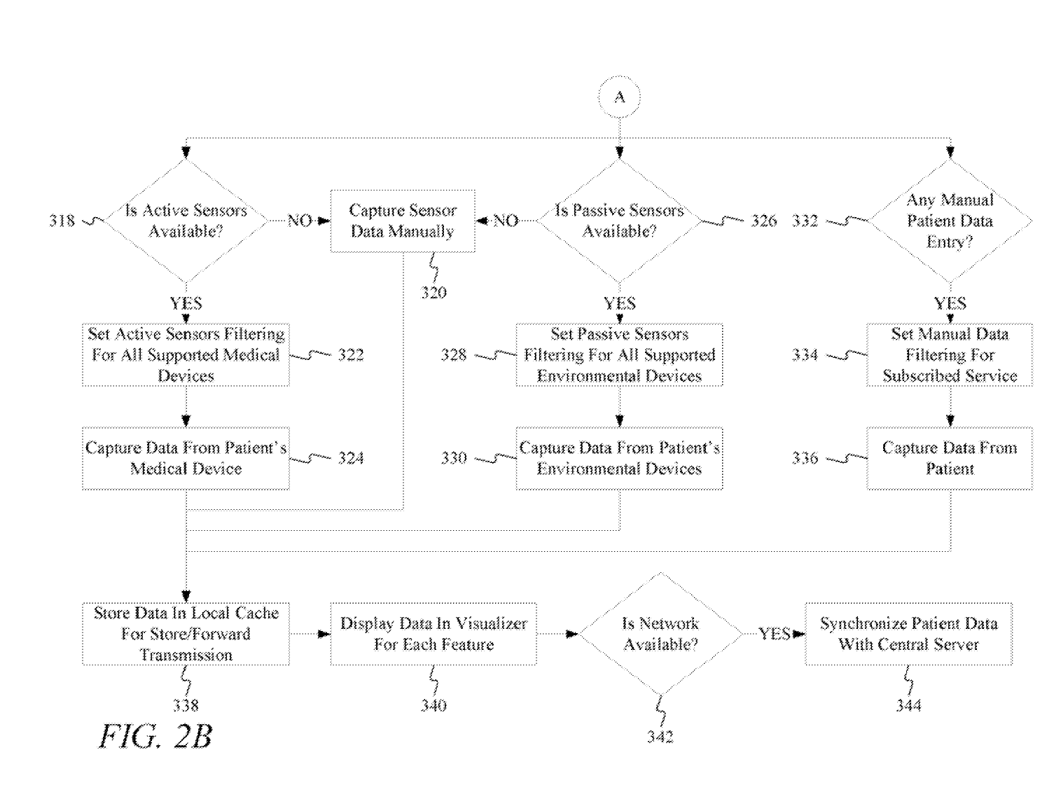

Consistent with an embodiment of the present invention, the aforementioned systems and methods for determining a medical plan that includes at least one self administered medical procedure are comprised of at least a patient health system and health care provider analysis system operatively coupled via a communications network. These systems and methods may be implemented by the embodiments of systems illustrated in FIGS. 1 and 2. Referring to FIG. 1, a system according to the first embodiment of the invention is shown. The system 100 supports communication between a patient data input terminal 101 and a central server 110 of a health care provider that is remote to the terminal 101 and operatively connected via communications medium 120. The patient terminal 101 is operatively connected to a camera 102, an audio transceiver 103, and an active sensor 104. The central server 110 is operatively connected to a video display 112 and an audio transceiver 113. The camera 102 is in communication with the video display 112 such that the video display 112 provides images in dependence upon video image information incident the camera 102. The audio transceivers 103 and 113 are shown as tethered devices. A person of skill in the art will appreciate that this need not be the case. For example, the audio transceiver 103 is optionally integrated into the terminal 101 and operates in a fashion analogous to a speakerphone. A variety of different communications infrastructure is optionally used as the communications medium 120. For example, the terminal 101 optionally communicates with the server 110 via a wireless link, an Internet link or a plain old telephone system (POTS).

In use, a communications path is established between a medical professional operatively connected to the central server 110 via a health care provider station 114 and a patient proximate the terminal 101. The camera 102 is disposed such that the patient is in view of the camera 102. The medical professional verbally requests the patient carry out a medical procedure in accordance with a predetermined medical plan associated with the patient. The patient does so in a fashion that supports verification by the medical professional. Clearly, if the patient is unwilling to follow the medical plan it is desirable to evaluate the medical plan and generate a more suitable alternative. The first embodiment of the invention seeks to provide sufficient information to the medical professional such that the medical professional is able to confidently verify that the patient is following their predetermined medical plan.

Clearly, the hardware used to ensure that the patient acts in accordance with their medical plan varies based upon what is done in order to carry out the medical plan. The first embodiment of the invention is believed to be highly beneficial for verifying that a patient follows a pharmaceutical prescription. Specifically, it is well known that the effects of certain psychiatric conditions are mitigated by certain prescriptions. Unfortunately, it is also the case that many psychiatric patients are unwilling to take their prescribed medications. Using the first embodiment of the invention, a medical professional operatively connected to the central server 110 via a health care provider station 114 and remote to the terminal 101 initiates communication with the patient. In accordance with a predetermined medical plan, the patient is requested to take a pill as part of their prescription. The patient presents the pill to the video camera 102 such that the medical professional is able to confidently identify it. The patient is then instructed to ingest the pill. The patient ingests the pill. The medical professional then optionally requests that the patient open their mouth to clearly show that the pill is not in their mouth. Further optionally, the patient shows the hand that held the pill before the pill was ingested. In this way the medical professional is able to confidently establish that the patient has swallowed the pill. Alternatively, if the patient does not demonstrate that they have taken their prescribed medication the medical professional is aware of the situation. In such cases it is apparent that the patient is not following the medical plan and that other measures are likely to be necessary in order to ensure that the patient follows the medical plan.

In an alternative embodiment, the invention includes a memory buffer 109 disposed electronically proximate the terminal 101. Specifically, the memory buffer 109 supports receiving a relatively large amount of data from the terminal 101 via a relatively high bandwidth interface. The memory buffer 109 is optionally located within the terminal 101. In use, the memory buffer 109 stores data indicative of a response of a patient data representative if patient health. A person of skill in the art will appreciate that the ability to support communications between remote locations is often difficult to achieve in practice, particularly in areas that are not well served. The memory buffer 109 serves to mitigate such problems by storing image data associated with the patient's responses. Specifically, when communication between the terminal 101 and the server 110 is poor, the data associated with the patient's response is stored in the memory buffer 109 and transferred to the server 110 in a gradual fashion that supports verification of the delivery of the data. In this way, should communication between the terminal 101 and the server 110 fail, the patient's response is still known. Optionally, the memory buffer 109 supports downloading of data stored therein via a local communications port, such as a universal serial bus (USB) port. In this way, if communications should fail for an extended period of time, the data stored within the memory buffer 109 is optionally downloaded proximate the terminal 101 and a transferred to the medical professional other than via communication between the terminal 101 and the server 110. Optionally, data stored within the memory buffer 109 is encrypted to inhibit unauthorized access to the data.

Further optionally, the terminal 101 comprises a computing device and a non-volatile storage medium. The non-volatile storage medium comprises predetermined medical instructions provided in accordance with a medical plan. When the patient accesses the terminal 101 they identify themselves. The computing device within the terminal interprets data within the non-volatile memory and provides the predetermined medical instructions to the patient in accordance with the data. The response of the patient is then stored in the memory buffer 109. Data within the memory buffer 109 is later transmitted to the server 110. Optionally, the server 110 comprises a memory that stores the transmitted data. This alternative embodiment is highly beneficial because it provides many of the benefits of the first embodiment of the invention absent the medical professional having to be available when then patient is requested to follow the instructions. Thus, if a patient is requested to take a blood pressure test at three o'clock in the morning it is not necessary to have a medical professional available to request the test. Clearly, a person of skill in the art will appreciate that a certain level of interaction between the medical professional and the patient is desirable in order to provide a high likelihood of the patient following the medical plan. Therefore, it is suggested that the degree of interaction between the medical professional and the patient be chosen in an appropriate fashion to support good compliance with the medical plan.

Optionally, the terminal 101 supports additional medical testing equipment, such as a heart rate monitor, a weight scale and a blood glucose meter. These instruments are designed to support providing measured health information to the server 110. A person of skill in the art will appreciate that the first embodiment of the invention is easily modified to support a wide variety of different medical and environmental sensors.

Alternatively, the terminal 101 includes a monitor 105 for displaying video and providing visual information. In use, the medical professional is able to provide video information to the patient. Thus, should the patient experience some difficulty with a self-administered medical procedure, the medical professional is able to provide the patient relevant instruction both visually and audibly in order to assist the patient. Optionally, the medical professional provides a predetermined media stream to the terminal 101 where the media stream comprises medical instruction information for the purpose of instructing a patient regarding a self-administered medical procedure. Further optionally, a set of such procedures are stored in a non-volatile storage memory proximate the server 110 and transferred as a media stream to the terminal 101. Advantageously by providing stored instructions the system supports providing same instructions to multiple patients thereby reducing the time and effort to provide those instructions.

A person of skill in the art will appreciate that a wide variety of techniques are available to support communication between the terminal 101 and the server 110. Clearly, the choice of the technologies used is dependent upon a variety of factors, many of which are outside the scope of the present invention. Further, a person of skill in the art will appreciate that the embodiments of the invention presented are intended to be illustrative of the invention and not limiting. Numerous other embodiments of the invention will be apparent to one of skill in the art.

Referring now to FIG. 2A, the patient station which is a remote device utilized to enter patient physiological data remotely, may be any one of the following devices: a tablet PC, a PDA, a personal computer, a Kiosk, laptop or any other computer-implemented configuration including a display screen, processor and memory. When operating a patient station, initially the device must be turned on 302. Upon activating the patient station, a communications link test is performed 304 by a communications link module to determine the network communication type across which the patient station shall transmit patient data. It is to be understood that the network communication type may be a wide area network that includes dialup (56k), ISDN, T1, DSL, broadband, cellular, satellite, or any other communications medium that facilitates the transmission of data. The communications link module that checks the network communication type performs an assessment of which communication types may be available and also selects the optimal communications network if more than one communications network type is detected. For example, it is contemplated that there may be patient stations that include both dialup and broadband network communications. The communications link module that checks the network communication type selects the optimal network communication type and then determines whether the communications network selected is available 306. If the network is not available, the communications link module sets up the patient station to operate in offline mode 308.

During offline mode 308 the patient may still use the patient station, even though there is no network communication between the patient station and the remote healthcare server that functions as a central data repository for patient information. However, the patient may interact with the patient station graphical user interface application to input data manually and to facilitate automatic capture of data from active and passive sensors. Data input during offline mode is locally cached. Offline mode also facilitates setting of security on patient data, configuration of encryption and data compression technology being used. Alternatively, if the communications network is available 306, the patient station sets parameters for transmitting data across the available communications network. The parameters that shall be set are determined by the network communication type. Next, the patient station determines the type of care plan services the patient has access to 310. The care plan services may include services such as video visit, vital signs monitoring, blood pressure monitoring, blood glucose monitoring, blood oxygen monitoring, body weight monitoring, body temperature monitoring, pulmonary function analysis, respiratory monitoring, neurological monitoring, cardiac monitoring, sleep monitoring bathroom visit monitoring, bedroom visit monitoring, activity monitoring (sensors in the house), meal preparation monitoring air quality monitoring, patient fall status monitoring (sensors to detect body up/down position) or any other services that may be available to a patient via the patient workstation. It is to be understood that the care plan services that are active as icons on the patient station shall be configured by the care provider remotely or directly upon the patient station prior to delivery. The patient station is configured for the patient based on the patient's illnesses and the services that a patient may require. For example, if a patient is diabetic, the patient station shall be configured to interface with a glucose meter and a weight scale and have the medication reminder service. By way of further example, if the patient is a cardiac heart failure patient (CHF), the patient station may be configured to interface with a stethoscope as well as an apparatus for capturing the patient's ECG measurements.

Following a determination by the patient workstation that the network is available, a determination is made by the patient station configuration module of the bandwidth for the communications network and the services which may be pushed on that bandwidth 310. Next the system sets the patient station up for user interface display 312. If the network communication type is dialup, a patient would not be able to facilitate wound management interface, because wound management interface includes a video component. If the network communication is high-speed DSL, wound management is an application which may be engaged because the video component may be streamed via the high-speed DSL connection. For example a patient having diabetes, may subscribe to the wound management service and thereby have an active wound management icon display on the patient station. The wound management service allows wounds to be displayed and recorded by the healthcare provider. Typically during operation, a patient station camera is utilized to facilitate capture of ulcers on the feet of the patient for transmission back to the central server of the healthcare provider system. The images are transmitted from the patient station back to the central server of the healthcare provider system. A nurse stationed at a work station which is connected to the central server may view the images to provide feedback which may be immediate when images are viewed as they are being streamed across the communications network. The images may also be viewed at a later time when the video images are stored in server memory.

Next, the patient station configuration module sets the parameters for user interface display, data encryption, data compression, and data access, authorization and consent 312. The data encryption parameters being utilized is a key pair encryption. A key that is stored on the healthcare provider's server is utilized to encrypt the data. Utilization of key pair encryption guarantees that data transmitted over the communication network cannot be intercepted and viewed by individuals intercepting data being transmitted over the communications network. Data compression is performed to facilitate shrinking of data so that the data can be transmitted on a network having very low bandwidth. For example if the communications network is dial-up, the data may be compressed and transmitted at a faster rate. The compression algorithm is a standard application protocol interface (API). Data access, authorization and consent is the control mechanism whereby the system dictates the individuals who have access to and can actually look at the patient data once it is captured. The data access, authorization and consent parameters define the individuals whom may have access to patient data. Data access, authorization and consent parameters are defined by the patient through the patient station. For example a patient may define the parameters such that his or her pharmacist does not have access to the patient's physiological data representative of the patient's vital signs. However, the pharmacist may have access to data concerning a patient's diet, medication plan and any other data which the patient determines that the pharmacist needs to have access.

Next, services to which the patient subscribes are loaded onto the patient station by loading the icons that correspond to a subscribed service onto the patient station 314. Based on the icons loaded onto the patient station, active and passive sensors that correspond to the service icons loaded may be activated by engaging the icons. For example, an icon is loaded onto the patient workstation in order to facilitate glucose monitoring. That icon has to be operatively connected to a sensor, which in this example is an active sensor, such as a glucose monitor. For glucose monitoring interface to be fully functional on the patient station, the glucose monitor must be activated and operatively connected to the patient workstation. In one embodiment operative connection and activation may be performed by Bluetooth communications. Next, parameters are set for active and passive sensors 316. Engaging the subscriber service icon causes the parameters for the active and passive sensors to be set 316. It is contemplated that active and passive sensors may be connected or communicating with the patient station via wired USB or serial connections, wireless Bluetooth, RFID or Zigbee communications or any other third party communications protocol. The Bluetooth communications link is performed by pairing the workstation with the active or passive sensor in accordance with normal Bluetooth pairing protocol.

Following the setup of the parameters for active and passive sensors, in accordance with the services associated with a patient, the system tries to determine whether any active or passive sensors are available 318, 326. In the case of a diabetic patient they have engaged the icon for measuring their blood sugar level through use of the glucose monitor, an active sensor. Upon a determination that there are active sensors 318, a filtering mechanism 322 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also associated with data that has been flagged and the alert is transmitted to the remote central server and thereby to previously defined individuals to provide notice that something abnormal is occurring with the patient or the active sensors.

Upon a determination that there are passive sensors 326, a filtering mechanism 328 is engaged to make sure that only the proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also associated with the data that has been flagged and the alert is transmitted to the remote central server and thereby to previously defined individuals to provide notice that something abnormal is occurring with the patient or the sensors.

The system is also capable of facilitating manual data entry 332. For example if a patient needs to enter their temperature into the patient station, because thermometers are not Bluetooth capable nor do they have USB or any other communications capability, the user must enter data representative of the patient's temperature into the patient station manually. The patient station includes a keypad whereby the patient may enter the value that the patient sees on the medical device. Following a determination that there is data for manual data entry 332, a filtering mechanism 334 is engaged to make sure that only proper data is being pulled into the patient workstation. Proper data is data that falls within previously defined minimum and maximum range levels. Data falling within the acceptable range is captured and stored on the patient station. When data received is above or below the range of acceptable data, the data is flagged and saved. An alert is also attached to the data and the alert is transmitted to the remote healthcare provider system to indicate a potential patient health issue or a problem with the device for which data has been entered.

The patient data captured by the patient station is stored in a local cache for the store forward transmission function 338. The store forward function defines how much of a data stream needs to be stored in order to facilitate safe data transmission in order to allow for the recovery of data which may have been lost during a faulty transmission or service interruption. For example, the amount of data that needs to be stored in the local cache before being forwarded depends on whether data is to be transmitted across a broadband connection network or a dial up connection.

In one embodiment, when the communications network is dial up, data is stored in 10 second groupings and forwarded. When the communications network is broadband, data packets are stored in 30 second blocks and forwarded. The 30 second block of data packets are transmitted across the communications network in an orderly fashion. Patient station data processing includes an algorithm that tracks the data packets being sent and includes a verification mechanism for verifying that all data packets transmitted within a 30 second block were received. The verification mechanism is the transmission of an acknowledgement that is sent back to the patient station from the central server following verification by the algorithm that the entire 30 second block of data packets was received. The algorithm determines whether a block of data packets has been received by the size of the block of data packets. For example a first 30 second block of data packets is created then sent, a second 30 second block of data packets is created then sent, a third 30 second block of data packets is created then sent and so on. This helps facilitate maintaining the integrity of the data so that if there is a connection loss during transmission of the second 30 second block of data packets, no other data shall be transmitted until the connection has been reestablished. Upon reestablishing the connection, the entire second 30 second block of data packets shall be sent again and a third 30 second block of data packets will then be sent behind the second 30 second block of data packets in the previously defined sequence.

Next, the data for each service is displayed in a visualizer to facilitate graphic representation of captured patient data 340. Next the system checks to determine if the communications network is online or available 342. If the network is available the patient workstation synchronizes and transmits patient data with the central server 344.

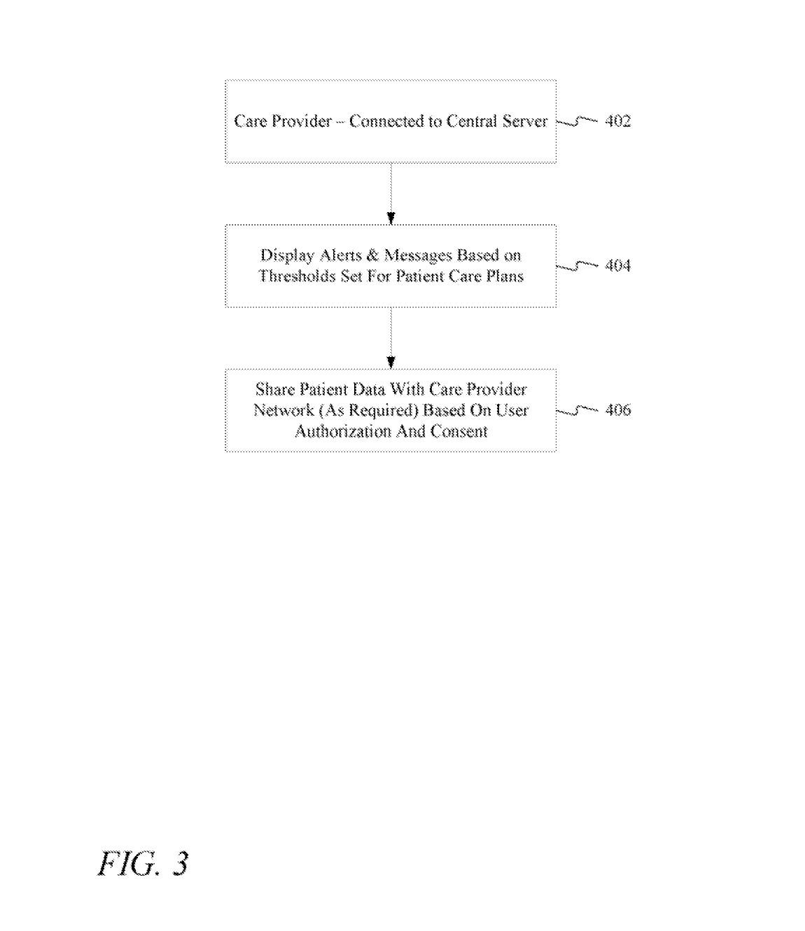

The central server 344 serves as a centralized data repository to which health care providers and other individuals who have been granted access authorization and consent by the patient to certain data files may connect and gain access to information to which they have authorization. As illustrated in FIG. 3, health care providers may connect to the central server 402. Connection may occur via WAN, but is generally done via a web based Internet connection. The application that manages connection to the host server is simply a web browser that individuals enter and gain access to in response to the entry of their respective credentials. Upon gaining access to the web browser, the user receives displays, alerts and messages based on their respective access authorization and consent previously defined by the patient 404. The web browser facilitates access to the centralized data repository by allowing users to login and gain access to files based on the authorization and consent provided a user by the patient 406. The health care provider seeking access to the central server may be a network of care providers including any of the following individuals: nurse, primary physician, pharmacist, family members, etc. These individuals each have access to certain subsets of the patient data based on the authority assigned at the access authorization and consent previously defined 406.

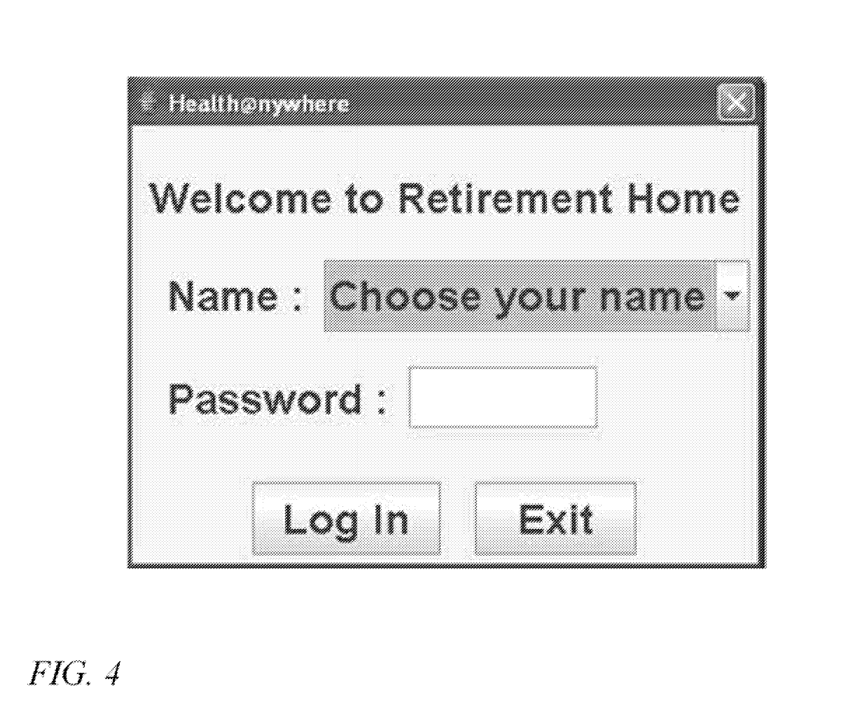

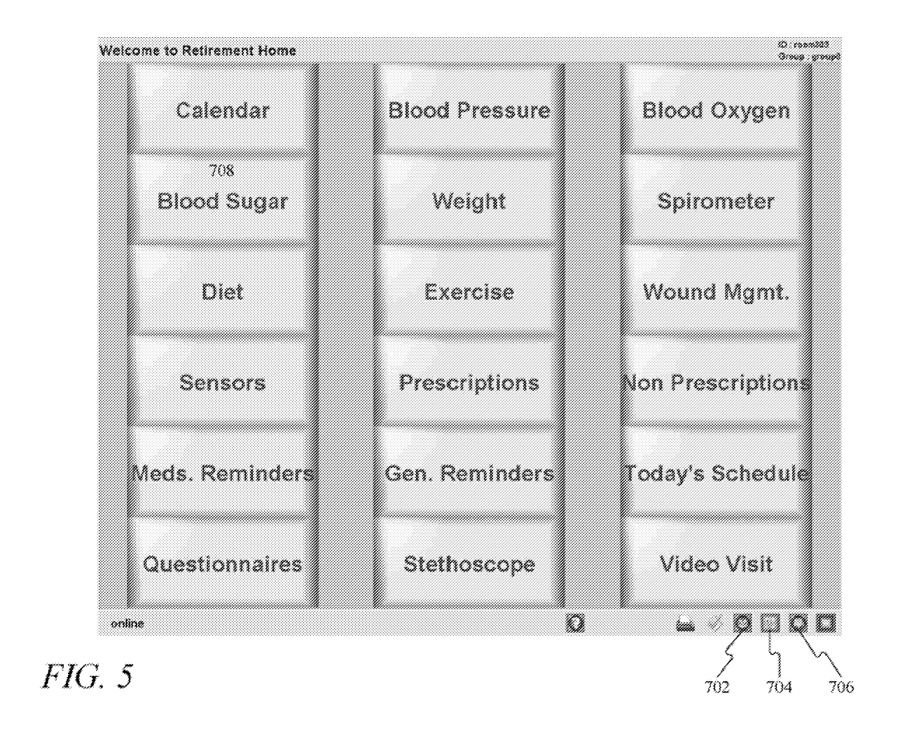

In an example of using systems and methods consistent with embodiments of the present invention to transmit data representative of a patient's environment from a patient health station to which a plurality of environmental sensors are operatively connected in order to facilitate automatic transmission of patient environment data, the patient engages the system by logging into the patient station. FIG. 4 illustrates a login screen 700, from which the patient logs onto a website with a secure login ID and password in order to create a session. Following login, the patient is allowed to access the welcome screen 710 illustrated in FIG. 5. The welcome screen illustrates the icons for each service to which a patient may subscribe. The icons that are active is controlled by the services that a patient requires as a result of an illness. The icon 702 is a link to a speech recognition application which may be turned on by engaging icon 702. Engaging icon 702 will actually activate an automatic speech recognition engine which allows the patient to order all the commands such as calendar, weight, diet, exercise, instead of by engaging the associated icons. Engaging icon 704 on the welcome screen will activate a status bar which may be used to change the font, the colors, and the backgrounds of the interface. Engaging icon 706 on the welcome screen will initiate a display box illustrating who you are and the server to which you are connected. The server to which the user is connected is important because in some instances the user may be connected to a healthcare provider server and in others the user may be connected to the main central host server.

Of the icons illustrated in FIG. 5, in most instances, all of these icons will never be turned on because most patients will not subscribe to ever service. The icons that are turned on depends on what disease a patient has and the services the patient has subscribed to. Subscription and service setup is performed by a care provided at a nurse station. For example, patient Smith is going to utilize the system. The first thing that happens is that a care provider sets up a profile for Smith on the nurse station. Following the creation of a patient profile and record on the nurse station, the profile is saved on the central server. Next the patient is provided with a patient station, for example a tablet PC, and upon activating the tablet it communicates with the server and pulls down the profile. The profile facilitates activation of respective icons and everything a care provider has set up for the patient at the nurse station. The patient station may be any kind of computing apparatus so long as it has a processor, memory and an input device.

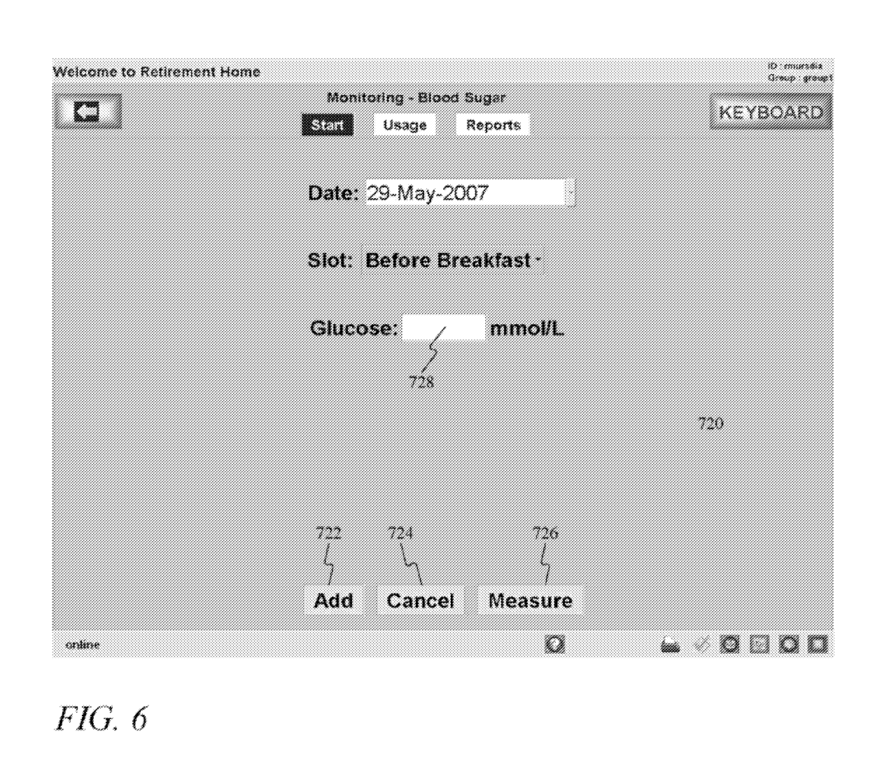

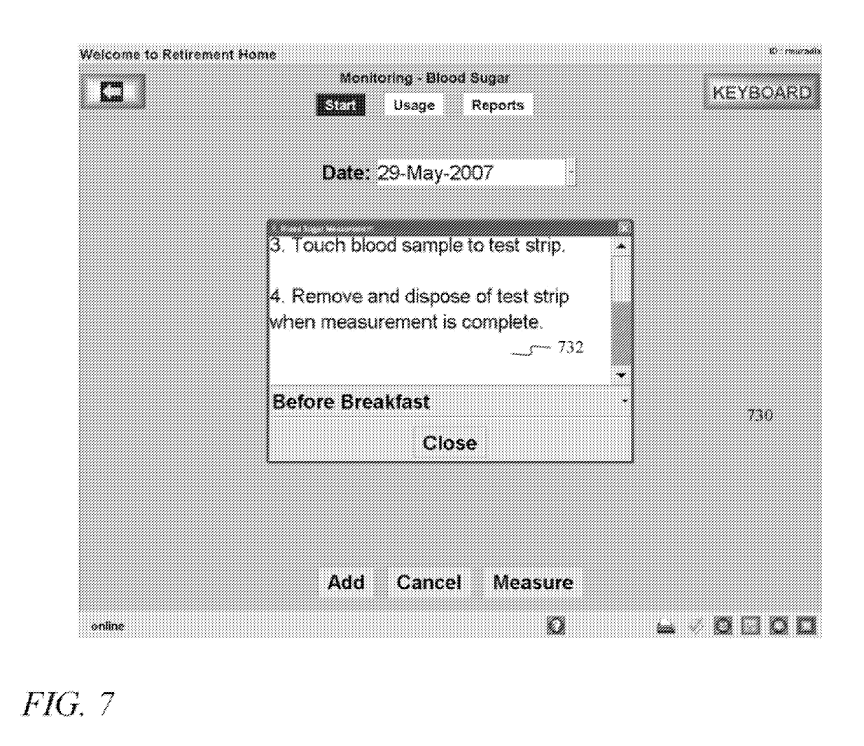

Upon initiating the blood sugar icon 708, the blood sugar monitoring screen 720 shown in FIG. 6 is illustrated. The blood sugar monitoring screen 720 provides 3 option, add 722, cancel 724, and measure 726. Upon engaging the measure icon 726, the blood sugar monitoring screen 730 that is displayed is illustrated in FIG. 7. This screen provides instructions on how to take a measurement 732. So with instructions written there, you can just play them back and hear them. Patient simply follows the instructions, and soon as blood sugar level is captured, it will be pushed on to the field 728 illustrated on the blood sugar monitoring screen 720 shown in FIG. 6. If the wireless link to the blood glucose monitor is not working, the user may alternatively initiate the keyboard button 729, which will cause a small keyboard to launch whereby the patient may read the glucose level and manually enter the data.

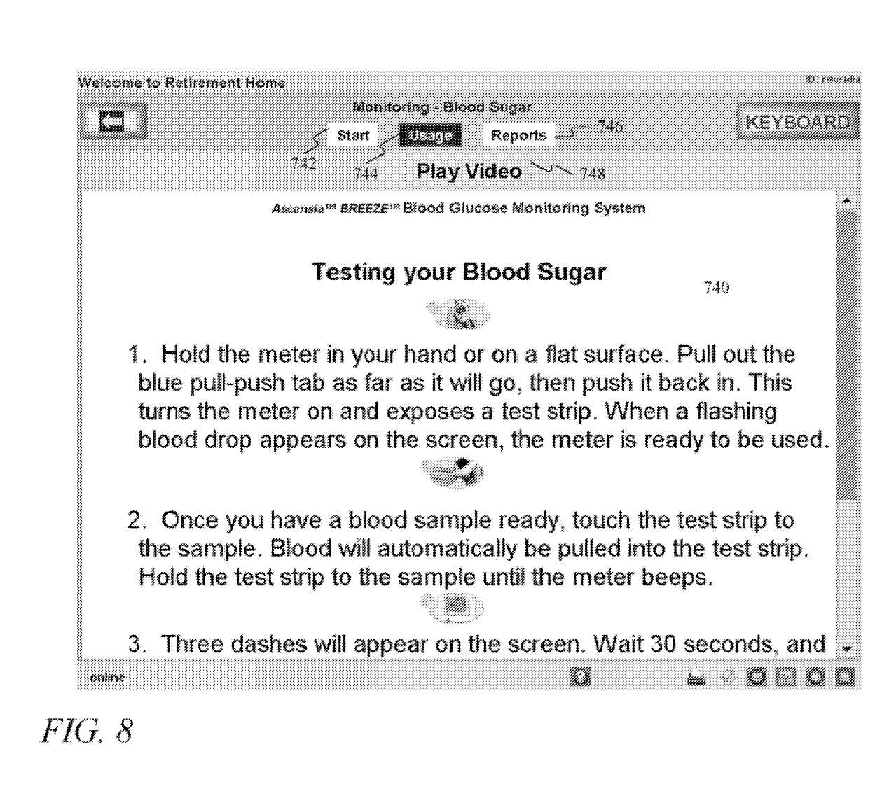

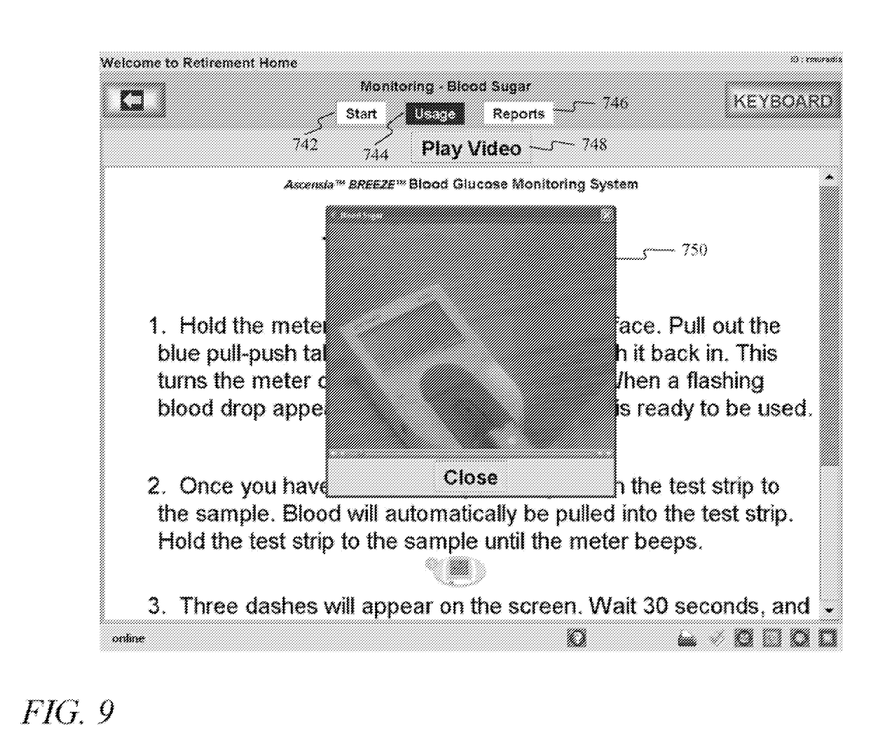

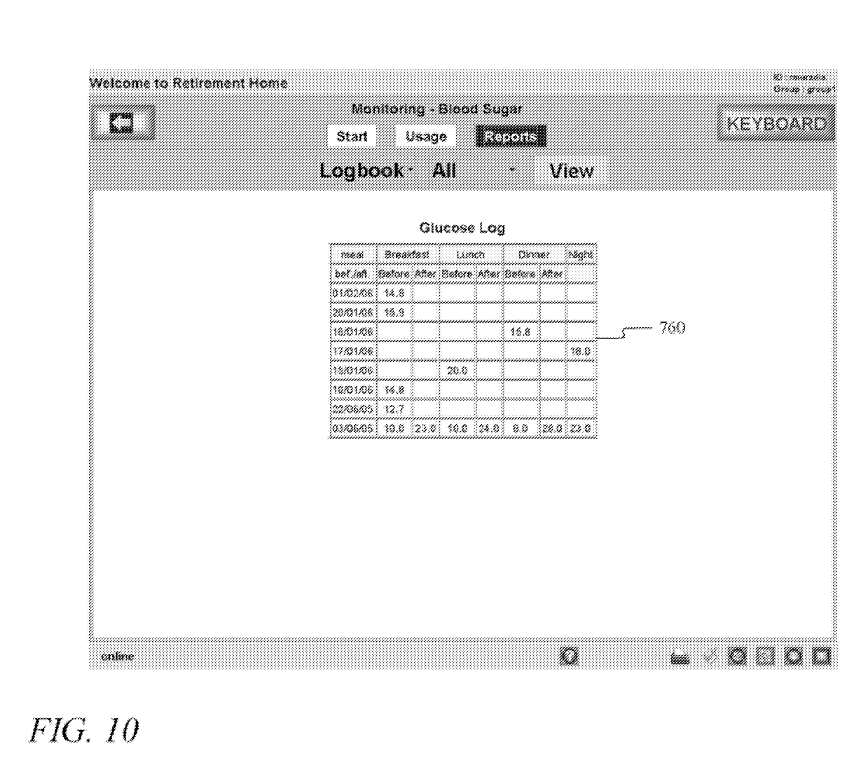

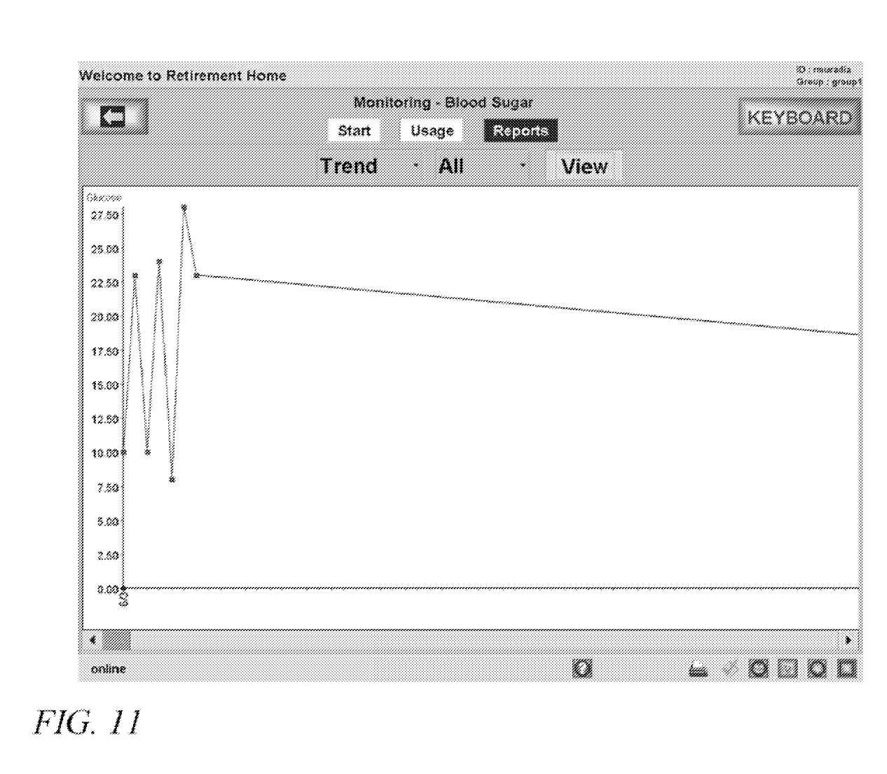

As illustrated in FIG. 8, if the patient presses the usage button 744 the instructions on the device and how it's to be use are presented. The patient may read the instructions or access video instruction by engaging the play video icon 748, which initiates a video box 750 illustrated in FIG. 9. Upon completion of gathering measurements, the patient may elect to have reports prepared and as illustrated in FIG. 10, the patient or care provider is allowed to review the patient's log book. FIG. 11 also illustrates the ability to graph the measurement in a chart.

While certain features and embodiments of the invention have been described, other embodiments of the invention will be apparent to those skilled in the art from consideration of the specification and practice of the embodiments of the invention disclosed herein. Furthermore, although embodiments of the present invention have been described as being associated with data stored in memory and other storage mediums, one skilled in the art will appreciate that these aspects can also be stored on or read from other types of computer-readable media, such as secondary storage devices, like hard disks, floppy disks, or a CD-ROM, a carrier wave from the Internet, or other forms of RAM or ROM. Further, the steps of the disclosed methods may be modified in any manner, including by reordering steps and/or inserting or deleting steps, without departing from the principles of the invention.

It is intended, therefore, that the specification and examples be considered as exemplary only, with a true scope and spirit of the invention being indicated by the following claims and their full scope of equivalents.

What is claimed is:

1. A method comprising:
determining a medical plan comprising at least a medical procedure that is self administered;
establishing a communications link between a medical professional and a patent remote to the medical professional, the communications link supporting the transmission of video information from the patient to the medical professional;
instructing the patient to initiate a self administered medical procedure in accordance with the medical plan;
receiving video information from the patient indicative of compliance with the instruction.

2. A method according to claim 1 comprising:
prior to establishing the communications link, determining that the patient is likely to be other than compliant with an instruction to initiate a self administered medical procedure in accordance with the medical plan;
upon receiving video information from the patient, requesting additional video information indicative of compliance with the instruction.

3. A method according to claim 1 comprising:
prior to receiving video information from the patient, storing the video information in a memory buffer.

4. A method according to claim 1 wherein establishing a communications link comprises:
the transmission of audio information from the medical professional to the patient.
